(19) United States
(12) Patent Application Publication
(10) Pub. No.: US 2012/0095341 A1
(43) Pub. Date: Apr. 19, 2012

(54) ULTRASONIC IMAGE PROCESSING APPARATUS AND ULTRASONIC IMAGE PROCESSING METHOD

(75) Inventors: Eiichi SHIKI, Otawara-Shi (JP); Kenji HAMADA, Otawara-Shi (JP); Takashi OGAWA, Nashishiobara-shi (JP)

(73) Assignees: TOSHIBA MEDICAL SYSTEMS CORPORATION, Otawara-Shi (JP); KABUSHIKI KAISHA TOSHIBA, Tokyo (JP)

(21) Appl. No.: 13/331,730

(22) Filed: Dec. 20, 2011

Related U.S. Application Data
(63) Continuation of application No. PCT/JP2011/073943, filed on Oct. 18, 2011.

(30) Foreign Application Priority Data
Oct. 19, 2010 (JP) 2010-234666

(51) Int. Cl. A61B 8/12; A61B 8/06

(52) U.S. Cl. 600/443

(57) ABSTRACT

An ultrasonic diagnostic apparatus according to an embodiment acquires first and second volume data by scanning a three-dimensional region including the lumen of an object in a B mode and a blood flow detection mode with ultrasonic waves, sets a viewpoint and a plurality of lines of sight with reference to the viewpoint in the lumen, determines a line of sight, of the plurality of lines of sight, on which data corresponding to an intraluminal region, tissue data corresponding to the outside of the lumen, and blood flow data outside the lumen are arranged. The apparatus controls at least a parameter value attached to each voxel of the tissue data existing on the determined line of sight. The apparatus generates a virtual endoscopic image based on the viewpoint by using the first volume data including voxels whose parameter values are controlled and the second volume data.

### CROSS REFERENCE TO RELATED APPLICATIONS

This application is a Continuation Application of PCT Application No. PCT/JP2011/073943, filed Oct. 18, 2011 and based upon and claiming the benefit of priority from prior Japanese Patent Application No. 2010-234666, filed Oct. 19, 2010, the entire contents of all of which are incorporated herein by reference.

### FIELD

Embodiments described herein relate generally to an ultrasonic diagnostic apparatus, ultrasonic image processing apparatus, and ultrasonic image processing method which can simultaneously capture a luminal image and a blood flow image near the lumen when performing three-dimensional image display in ultrasonic image diagnosis.

### BACKGROUND

An ultrasonic diagnostic apparatus is designed to apply ultrasonic pulses generated from vibration elements provided on an ultrasonic probe into an object and acquire biological information by receiving reflected ultrasonic waves caused by acoustic impedance differences in the tissue of the object through the vibration elements. This apparatus can display image data in real time by simple operation of bringing the ultrasonic probe into contact with the body surface. For this reason, the apparatus is widely used for morphological diagnosis and functional diagnosis of various kinds of organs.

Recently, in particular, it is possible to perform more advanced diagnosis and treatment by generating three-dimensional image data, MRP (Multi-Planar Reconstruction) image data, and the like using the three-dimensional data (volume data) acquired by three-dimensional scanning by a method of mechanically moving an ultrasonic probe on which a plurality of vibration elements are one-dimensionally arranged or a method using an ultrasonic probe on which a plurality of vibration elements are two-dimensionally arranged.

On the other hand, there has been proposed a method of making an observer virtually set his/her viewpoint and line-of-sight direction in a hollow organ represented by the volume data obtained by three-dimensional scanning on an object and observe the inner surface of the hollow organ from the set viewpoint as virtual endoscopic image (or fly-through image) data. This method can generate and display endoscopic image data based on the volume data acquired from the outside of an object, and can greatly reduce the degree of invasiveness to the object at the time of examination. This method allows to arbitrarily set a viewpoint and a line-of-sight direction with respect to a hollow organ such as a digestive canal or blood vessel in which an endoscope is difficult to be inserted, and hence can perform accurate examination safely and efficiently, which could not be performed by conventional endoscopes.

It is required to simultaneously observe a blood flow near the canal wall buried in the tissue in a virtual endoscopic image. Currently, an ultrasonic diagnostic apparatus which simultaneously displays a three-dimensional B-mode image and a three-dimensional image of a blood vessel has been in practical use. This apparatus allows to concatenate and display a three-dimensional B-mode image and a three-dimensional image of a blood flow or superimpose and display a three-dimensional B-mode image and a three-dimensional image of a blood flow upon making them translucent.

### CITATION LIST

### Patent Literature

Patent Literature 1: Jpn. Pat. Appln. KOKAI Publication No. 2005-110973

### BRIEF DESCRIPTION OF THE DRAWINGS

FIG. 1 is a block diagram showing the arrangement of an ultrasonic diagnostic apparatus 1 according to an embodiment.

FIG. 2 is a flowchart showing a procedure for near-lumen blood flow extraction processing.

FIG. 3 is a view for explaining the processing of setting a viewpoint, view volume, and line of sight.

FIG. 4 is a view for explaining the processing of setting a viewpoint, view volume, and line of sight.

FIG. 5 is a view for explaining data arrangement order determination processing in a case in which a line of sight extends through a blood flow in the tissue near the canal wall.

FIG. 6 is a view for explaining volume rendering processing in a case in which a line of sight extends through a blood flow in the tissue near the canal wall.

FIG. 7 is a view showing an example of the display form of a virtual endoscopic image including a blood flow near the canal wall buried in the tissue.

FIG. 8 is a view for explaining near-lumen blood flow extraction processing in a case in which color data behind the first B-mode data is at a position sufficiently spaced apart from the canal wall.

FIG. 9 is a view for explaining near-lumen blood flow extraction processing in a case in which color data behind the first B-mode data is at a position sufficiently spaced apart from the canal wall.

FIG. 10 is a view for explaining near-lumen blood flow extraction processing in a case in which no blood flow exists on a line of sight.

FIG. 11 is a view for explaining near-lumen blood flow extraction processing in a case in which a blood flow exists in the lumen.

FIG. 12 is a view for explaining near-lumen blood flow extraction processing in a case in which a blood flow exists in the lumen.

### DETAILED DESCRIPTION

In general, according to one embodiment, an ultrasonic diagnostic apparatus comprises a volume data acquisition unit configured to acquire first volume data corresponding to a three-dimensional region including a lumen of an object by scanning the three-dimensional region in a B mode with an ultrasonic wave and acquire second volume data by scanning the three-dimensional region in a blood flow detection mode with an ultrasonic wave, a setting unit configured to set a viewpoint in the lumen, and a plurality of lines of sight with reference to the viewpoint, a determination unit configured to determine a line of sight, on which tissue data corresponding to an outside of the lumen, and on which blood flow data corresponding to an outside of the lumen are arranged, a control unit configured to control at least a parameter value corresponding to each voxel of the tissue data existing on the determined line of sight, an image generation unit configured to generate a virtual endoscopic image based on the viewpoint by using the first volume data including voxels whose parameter values are controlled and the second volume data and a display unit configured to display the virtual endoscopic image.

Embodiments will be described below with reference to the accompanying drawings. Note that the same reference numerals in the following description denote constituent elements having almost the same functions and arrangements, and a repetitive description will be made only when required.

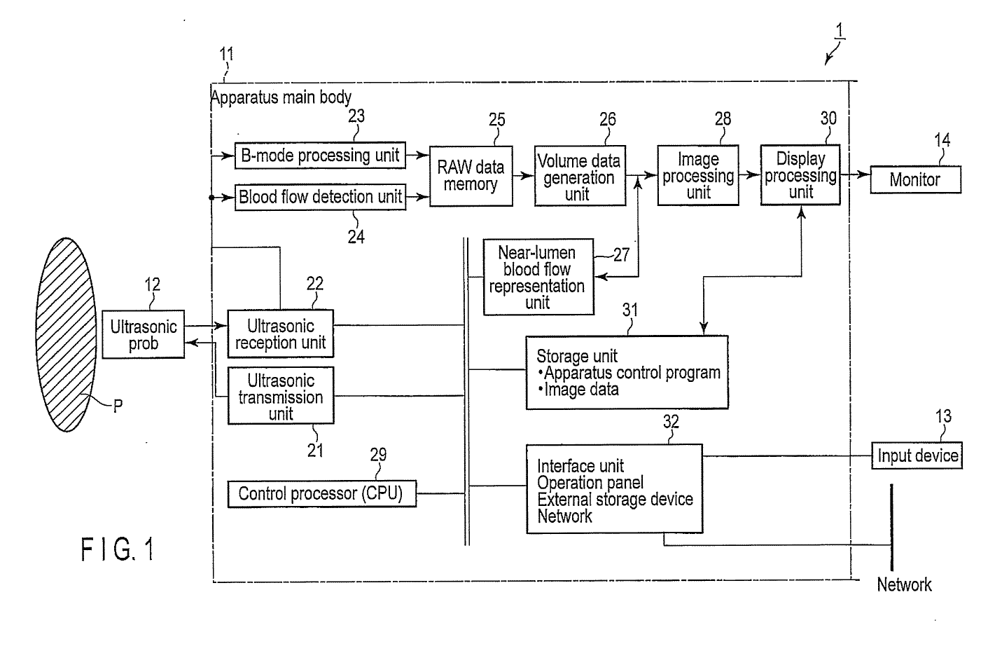

FIG. 1 is block diagram showing the arrangement of an ultrasonic diagnostic apparatus 1 according to this embodiment. As shown in FIG. 1, the ultrasonic diagnostic apparatus 1 includes an ultrasonic probe 12, an input device 13, a monitor 14, an ultrasonic transmission unit 21, an ultrasonic reception unit 22, a B-mode processing unit 23, a blood flow detection unit 24, a RAW data memory 25, a volume data generation unit 26, a near-lumen blood flow extraction unit 27, an image processing unit 28, a control processor (CPU) 29, a display processing unit 30, a storage unit 31, and an interface unit 32. The function of each constituent element will be described below.

The ultrasonic probe 12 is a device (probe) which transmits ultrasonic waves to an object and receives reflected waves from the object based on the transmitted ultrasonic waves. The ultrasonic probe 12 has, on its distal end, an array of a plurality of piezoelectric transducers, a matching layer, a backing member, and the like. Each of the piezoelectric transducers transmits an ultrasonic wave in a desired direction in a scan region based on a driving signal from the ultrasonic transmission unit 21 and converts a reflected wave from the object into an electrical signal. The matching layer is an intermediate layer which is provided for the piezoelectric transducers to make ultrasonic energy efficiently propagate. The backing member prevents ultrasonic waves from propagating backward from the piezoelectric transducers. When the ultrasonic probe 12 transmits an ultrasonic wave to an object P, the transmitted ultrasonic wave is sequentially reflected by a discontinuity surface of acoustic impedance of internal body tissue, and is received as an echo signal by the ultrasonic probe 12. The amplitude of this echo signal depends on an acoustic impedance difference on the discontinuity surface by which the echo signal is reflected. The echo produced when a transmitted ultrasonic pulse is reflected by the surface of a moving blood flow is subjected to a frequency shift depending on the velocity component of the moving body in the ultrasonic transmission/reception direction due to the Doppler effect.

Note that the ultrasonic probe 12 according to this embodiment is a two-dimensional array probe (a probe having a plurality of ultrasonic transducers arranged in a two-dimensional matrix) or a mechanical 4D probe (a probe which can perform ultrasonic scanning while mechanically swinging a piezoelectric transducer array in a direction perpendicular to the array direction), as a probe which can acquire volume data. However, the ultrasonic probe to be used is not limited to these examples. For example, it is possible to use a one-dimensional array probe as the ultrasonic probe 12 and acquire volume data by performing ultrasonic scanning while manually swinging the probe.

The input device 13 is connected to an apparatus body 11 and includes various types of switches, buttons, a trackball, a mouse, and a keyboard which are used to input, to the apparatus body 11, various types of instructions, conditions, an instruction to set a region of interest (ROI), various types of image quality condition setting instructions, and the like from an operator. The input device 13 also includes, for the near-lumen blood flow extraction function (to be described later), a dedicated switch for inputting a diagnosis region, a dedicated knob for controlling the range of color data used for visualization, and a dedicated knob for controlling the transparency (opacity) of a voxel.

The monitor 14 displays morphological information and blood flow information in the living body as images based on video signals from the display processing unit 30.

The ultrasonic transmission unit 21 includes a trigger generation circuit, delay circuit, and pulser circuit (none of which are shown). The trigger generation circuit repetitively generates trigger pulses for the formation of transmission ultrasonic waves at a predetermined rate frequency fr Hz (period: 1/fr sec). The delay circuit gives each trigger pulse a delay time necessary to focus an ultrasonic wave into a beam and determine transmission directivity for each channel. The pulser circuit applies a driving pulse to the probe 12 at the timing based on this trigger pulse.

The ultrasonic transmission unit 21 has a function of instantly changing a transmission frequency, transmission driving voltage, or the like to execute a predetermined scan sequence in accordance with an instruction from the control processor 29. In particular, the function of changing a transmission driving voltage is implemented by a linear amplifier type transmission circuit capable of instantly switching its value or a mechanism of electrically switching a plurality of power supply units.

The ultrasonic reception unit 22 includes an amplifier circuit, A/D converter, delay circuit, and adder (none of which are shown). The amplifier circuit amplifies an echo signal received via the probe 12 for each channel. The A/D converter converts the amplified analog echo signals into digital echo signals. The delay circuit gives each echo signal converted into a digital signal the delay time required to determine reception directivity and perform reception dynamic focusing. The adder then perform addition processing. This addition processing will enhance a reflection component from a direction corresponding to the reception directivity of the echo signal to form a composite beam for ultrasonic transmission/reception in accordance with the reception directivity and transmission directivity.

The B-mode processing unit 23 receives an echo signal from the ultrasonic reception unit 22, and performs logarithmic amplification, envelope detection processing, and the like for the signal to generate data whose signal intensity is expressed by a brightness level.

The blood flow detection unit 24 extracts a blood flow signal from the echo signal received from the reception unit 22, and generates blood flow data. In general, CFM (Color Flow Mapping) is used for blood flow extraction. In this case, the blood flow detection unit 24 analyzes a blood flow signal to obtain an average velocity, variance, power, and the like as blood flow data at multiple points.

The RAW data memory 25 generates B-mode RAW data as B-mode data on three-dimensional ultrasonic scanning lines by using a plurality of B-mode data received from the B-mode processing unit 23. The RAW data memory 25 generates blood flow RAW data as blood flow data on three-dimensional ultrasonic scanning lines by using a plurality of blood flow data received from the blood flow detection unit 24. For the purpose of reducing noise and improving image concatenation, it is possible to perform spatial smoothing by inserting a three-dimensional filter after the RAW data memory 25.

The volume data generation unit 26 generates B-mode volume data from the B-mode RAW data received from the RAW data memory 25 by executing RAW/voxel conversion. The volume data generation unit 26 performs this RAW/voxel conversion to generate B-mode voxel data on each line of sight in a view volume used in the near-lumen blood flow extraction function (to be described later) by performing interpolation processing in consideration of spatial position information. Likewise, the volume data generation unit 26 generates blood flow volume data on each line of sight in the view volume from the blood flow RAW data received from the RAW data memory 25 by executing RAW/voxel conversion.

The near-lumen blood flow extraction unit 27 executes each process according to the near-lumen blood flow extraction function (to be described later) for the volume data generated by the volume data generation unit 26 under the control of the control processor 29.

The image processing unit 28 performs predetermined image processing such as volume rendering, multi planar reconstruction (MPR), and maximum intensity projection (MIP) for the volume data received from the volume data generation unit 26 and the near-lumen blood flow extraction unit 27. In processing according to the near-lumen blood flow extraction function (to be described later), in particular, when information indicating a transparency is input or the transparency is changed via the input device 13, the image processing unit 28 executes volume rendering by using the opacity corresponding to the input or changed transparency. Note that an opacity is a reverse concept to a transparency. If, for example, the transparency changes from 0 (perfect opacity) to 1 (perfect transparency), the opacity changes from 1 (perfect opacity) to 0 (perfect transparency). Assume that this embodiment uses the terms “opacity” and “transparency”, respectively, in connection with rendering processing and the user interface.

Note that for the purpose of reducing noise and improving image concatenation, it is possible to perform spatial smoothing by inserting a two-dimensional filter after the image processing unit 28.

The control processor 29 has a function as an information processing apparatus (computer), and controls the operation of this ultrasonic diagnostic apparatus. The control processor 29 reads out a dedicated program for implementing the near-lumen blood flow extraction function (to be described later) from the storage unit 31, expands the program in the memory, and executes computation/control and the like associated with various kinds of processes.

The display processing unit 30 executes various kinds of processes associated with a dynamic range, brightness, contrast, γ curve correction, RGB conversion, and the like for various kinds of image data generated/processed by the image processing unit 28.

The storage unit 31 stores a dedicated program for implementing the near-lumen blood flow extraction function (to be described later), diagnosis information (patient ID, findings by doctors, and the like), a diagnostic protocol, transmission/reception conditions, a program for implementing a speckle removal function, a body mark generation program, a conversion table for setting the range of color data used for visualization in advance for each diagnosis region, and other data. The storage unit 31 is also used to store images in an image memory (not shown), as needed. It is possible to transfer data in the storage unit 31 to an external peripheral device via the interface unit 32.

The interface unit 32 is an interface associated with the input device 13, a network, and a new external storage device (not shown). The interface unit 32 can transfer data such as ultrasonic images, analysis results, and the like obtained by this apparatus to another apparatus via a network.

### Near-Lumen Blood Flow Extraction Function

The near-lumen blood flow extraction function of the ultrasonic diagnostic apparatus 1 will be described next. This function properly visualizes a blood flow near the canal wall buried in the tissue in a virtual endoscopic image. The function is designed to visualize the lumen of an organ or blood vessel as a diagnosis target (cyst or lumen) in the form of a virtual endoscopic image. For the sake of a concrete description, however, this embodiment assumes that the lumen is set as a diagnosis target, and a blood flow exists in the tissue near the canal wall. In this embodiment, the term “lumen” represents a cavity, a internal blood flow or a characteristic part of a tubular organ such as a blood vessel or a digestive canal. The embodiment will exemplify a case in which the color data (velocity, variance, power, and the like) captured in the CFM mode is used as blood flow data. However, the embodiment is not limited to this case. For example, it is possible to use blood flow data captured by using a contrast medium. Blood flow data using a contrast medium can be acquired by executing B-mode processing for an extracted blood flow signal using a harmonic method for the extraction of a blood flow signal.

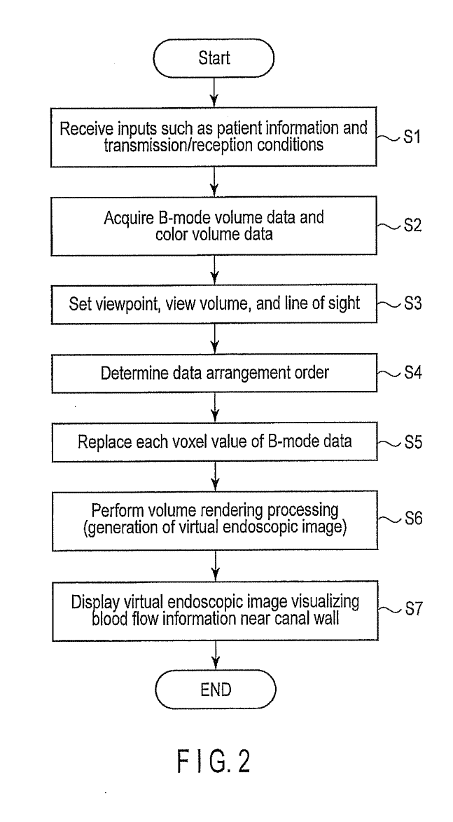

FIG. 2 is a flowchart showing a procedure for this near-lumen blood flow extraction processing. The contents of processing in each step will be described below.

### [Patient Information: Reception of Transmission/Reception Conditions as Inputs: Step S1]

The operator inputs patient information and selects transmission/reception conditions (a field angle for determining the size of a region to be scanned, a focal position, a transmission voltage, and the like), an imaging mode for ultrasonic scanning on a predetermined region of an object, a scan sequence, and the like via the input device 13 (step S1). The apparatus automatically stores the input and selected various kinds of information and conditions in the storage unit 31.

### [Acquisition of B-Mode Volume Data and Color Volume Data: Step S2]

The ultrasonic probe 12 is brought into contact with the body surface of the object to execute simultaneous ultrasonic scanning in the B mode and the CFM mode with respect to a three-dimensional region including the diagnosis region (the lumen in this case) as a region to be scanned. The B-mode processing unit 23 receives the echo signal acquired by ultrasonic scanning in the B mode via the ultrasonic reception unit 22. The B-mode processing unit 23 generates a plurality of B-mode data by executing logarithmic amplification, envelope detection processing, and the like. The blood flow detection unit 24 receives the echo signal acquired by ultrasonic scanning in the CFM mode via the ultrasonic reception unit 22. The blood flow detection unit 24 extracts a blood flow signal by CFM, and obtains blood flow information such as an average velocity, variance, and power at multiple points, thereby generating color data as blood flow data.

The RAW data memory 25 generates B-mode RAW data by using a plurality of B-mode data received from the B-mode processing unit 23, and also generates color RAW data by using a plurality of color data received from the blood flow detection unit 24. The volume data generation unit 26 generates B-mode volume data and color volume data by performing RAW/voxel conversion of the B-mode RAW data and the color RAW data (step S2).

Note that this embodiment has exemplified the case in which B-mode data and color data are generally acquired by simultaneous scanning. However, the embodiment is not limited to this. It is possible to acquire B-mode volume data and color volume data constituted by voxels whose positions have been associated with each other, by acquiring B-mode and color data at different timings and spatially positioning them afterward.

### [Setting of Viewpoint, View Volume, and Line of Sight: Step S3]

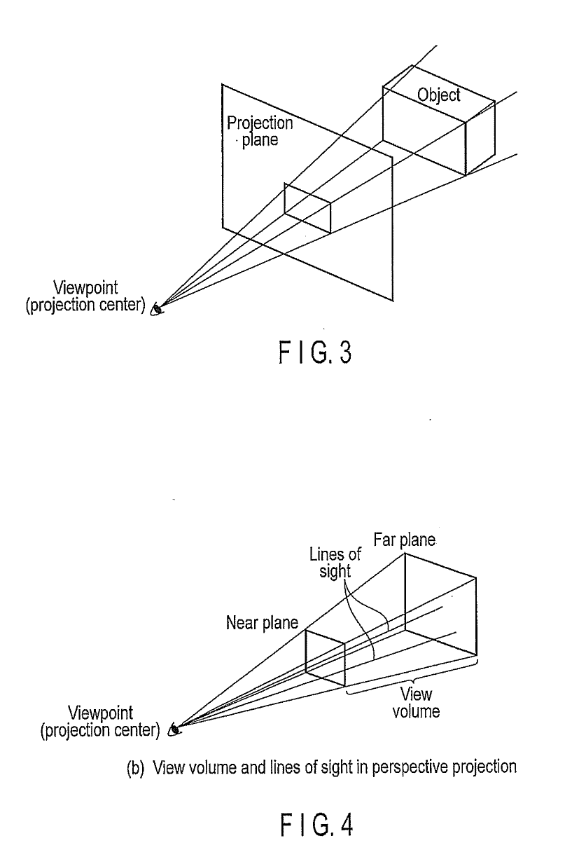

The near-lumen blood flow extraction unit 27 then sets three-dimensional orthogonal coordinates, viewpoint, view volume, and line of sight for the formation of a virtual endoscopic image by perspective projection like that shown in FIG. 3 with respect to the B-mode volume data and the color volume data (step S3). Note that the perspective projection method is a projection method in which a viewpoint (projection center) is set at a finite length from an object. This method is suitable for the observation of the canal wall because the larger the distance, the smaller the object looks. Assume that a viewpoint is set in the lumen. As shown in FIG. 4, a view volume is a region (to be visualized) where an object is seen when viewed from a viewpoint, and is also a region overlapping at least part of an ROI (Region Of Interest). A line of sight is each of a plurality of straight lines extending from the viewpoint in the respective directions in the view volume. B-mode data and color data on each line of sight are superimposed for each line of sight, and the resultant data is stored for each line of sight in a line-of-sight data memory (not shown) in the near-lumen blood flow extraction unit 27.

### [Determination of Arrangement Order of Data: Step S4]

Voxel data existing at each point on each line of sight stored in the line-of-sight data memory is considered to correspond to either of three data, namely void data (data corresponding to a void), B-mode data, and color data. The near-lumen blood flow extraction unit 27 determines the arrangement order of void data, B-mode data, and color data and the position information of color data when viewed from each viewpoint on each line of sight (step S4).

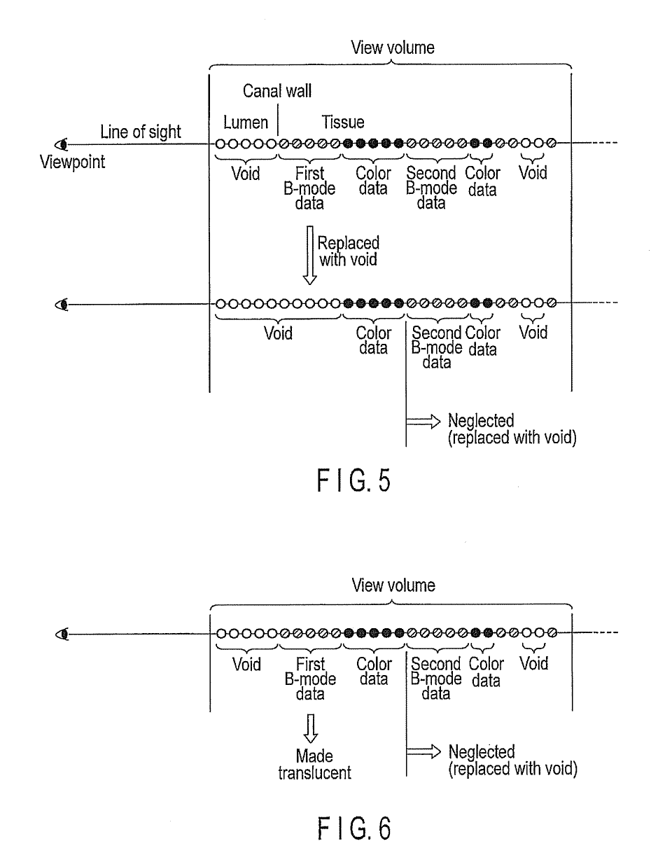

Assume that a given line of sight extends through a blood flow in the tissue near the canal wall. In this case, as indicated by the upper stage of FIG. 5, the respective data are arranged in the order of void data, B-mode data, color data, and B-mode data (for the sake of convenience, B-mode data adjacent to void data will be referred to as “first B-mode data”, and other B-mode data will be referred to as “second B-mode data”). The near-lumen blood flow extraction unit 27 can determine the arrangement order of void data, B-mode data, and color data when viewed from a viewpoint based on the distance from the viewpoint in each voxel obtained from the three-dimensional position information of each voxel on the line of sight and the position information of the viewpoint. The near-lumen blood flow extraction unit 27 also determines the position information of the first color data, which appears when tracing from the viewpoint along the line of sight, by using this arrangement order information.

When, for example, each point on a line of sight is set as three-dimensional orthogonal coordinates with a viewpoint serving as the origin, the absolute values of X-, Y-, and Z-coordinates of the point increase with the distance from the viewpoint. In this case, therefore, it is easy to determine the arrangement order of data from the values of the coordinates of each point on the line of sight.

### [Replacement of Each Voxel Value of B-Mode Volume Data: Step S5]

The near-lumen blood flow extraction unit 27 controls at least a parameter value attached to each voxel of tissue data (step S5). That is, as indicated by the lower stage in FIG. 5, the near-lumen blood flow extraction unit 27 zeroizes the parameter value (opacity) (or removing it by clipping processing) attached to each voxel of B-mode data (first B-mode data) located nearer to the viewpoint than the color data whose position information has been determined in step S4, thereby replacing each voxel value with void data. This makes the color data exist immediately behind the void data on each line of sight.

Note that the parameter value attached to each voxel indicates an opacity in this embodiment, as described above. However, the embodiment is not limited to this. For example, it is possible to use a voxel value, transparency, brightness, luminance, or color value as a parameter value. In addition, it is possible to directly execute control of the parameter value attached to each voxel in this step with reference to, for example, the correspondence relationship between the opacities and the voxel values of the respective voxels, assuming that the voxel values are attached to the respective voxels. Alternatively, it is possible to indirectly execute such control with reference to the correspondence relationship between brightnesses and the voxel values of the respective voxels and the correspondence relationship between brightnesses and opacities.

### [Volume Rendering Processing: Step S6]

The image processing unit 28 executes volume rendering by using the volume data in the view volume obtained by zeroizing the opacity of each voxel of the first B-mode data. In the case shown in FIG. 5, the second B-mode data exists behind (in the depth direction) the color data. It is therefore preferable from the viewpoint of an improvement in visibility to execute rendering by using only color data upon invalidating the opacities of the respective voxels of data behind the second B-mode data by replacing the opacities with void data by zeroizing the opacities (or removing the opacities by clipping processing). This makes it possible to obtain only a blood flow image of a region near the canal wall and generate, as a virtual endoscopic image, a volume rendering image obtained by visualizing blood flow information near the canal wall.

Alternatively, for example, as shown in FIG. 6, it is possible to execute rendering by making the first B-mode data translucent (setting the opacity of the B-mode data between 0 and 1). In this case, opacity=1 indicates perfect opacity, and opacity=0 indicates perfect transparency.

### [Display of Virtual Endoscopic Image Obtained by Visualizing Blood Flow Information Near Lumen: Step S7]

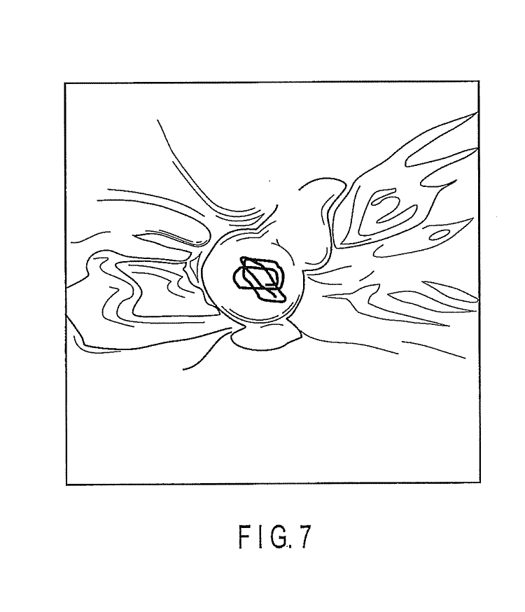

The monitor 14 displays the generated virtual endoscopic image including the blood flow near the canal wall buried in the tissue in, for example, the form shown in FIG. 7 (step S7). The observer can visually recognize the positional relationship between a morbid region and a blood flow near the canal wall easily and quickly by observing the displayed virtual endoscopic image.

### First Modification

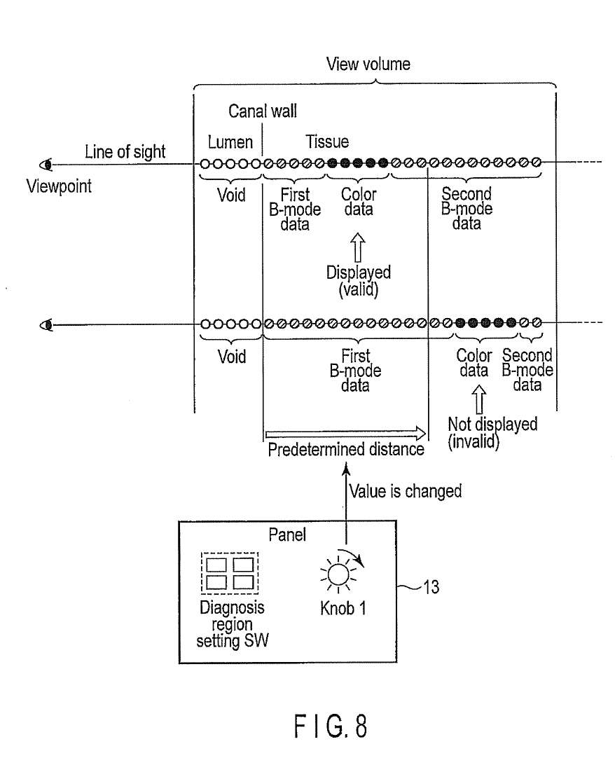

The above embodiment has exemplified the case in which the color data behind the first B-mode data is located near the canal wall, as indicated by the upper stage in FIG. 8. It can also be assumed that the color data behind the first B-mode data is at a position sufficiently spaced apart from the canal wall. In this case, in the processing in step S4 described above, as indicated by the lower stage in FIG. 8, it is possible to limit the range of color data to be visualized to a predetermined distance from the canal wall while displaying no color data located at a distance longer than the predetermined distance by invalidating the data. When invalidating distant color data in this manner, the apparatus performs volume rendering by using the first B-mode data, and replaces the color data and the second B-mode data behind the first B-mode mode with void data. In this case, it is preferable to obtain a predetermined distance from the canal wall in the vertical direction. It is however possible to simply validate color data at a predetermined distance from the start of the first B-mode data on a line of sight.

In addition, the apparatus can automatically set a distance from the canal wall, which defines the range of color data to be used for visualization, by using a conversion table in which the distance is set in advance for each diagnosis region. Furthermore, it is possible to change the distance from the canal wall to an arbitrary value by manual operation using the knob of the input device 13. When using the conversion table, if the operator selects a predetermined region with a diagnosis region setting switch (SW) as shown in FIG. 8, the near-lumen blood flow extraction unit 27 determines the range of color data to be visualized by determining a predetermined distance from the canal wall based on the selected region and the conversion table, and replaces the color data outside the distance range and the second B-mode data with void data. The image processing unit 28 executes volume rendering by using the volume data in the view volume after the replacement processing. When changing the distance from the canal wall by using the knob of the input device 13, if the operator changes the predetermined distance from the canal wall by using the knob like that shown in FIG. 8, the near-lumen blood flow extraction unit 27 determines the range of color data to be visualized by using the changed predetermined distance from the canal wall, and replaces the color data outside the distance range and the second B-mode data with void data. The image processing unit 28 executes volume rendering by using the volume data in the view volume after the replacement processing.

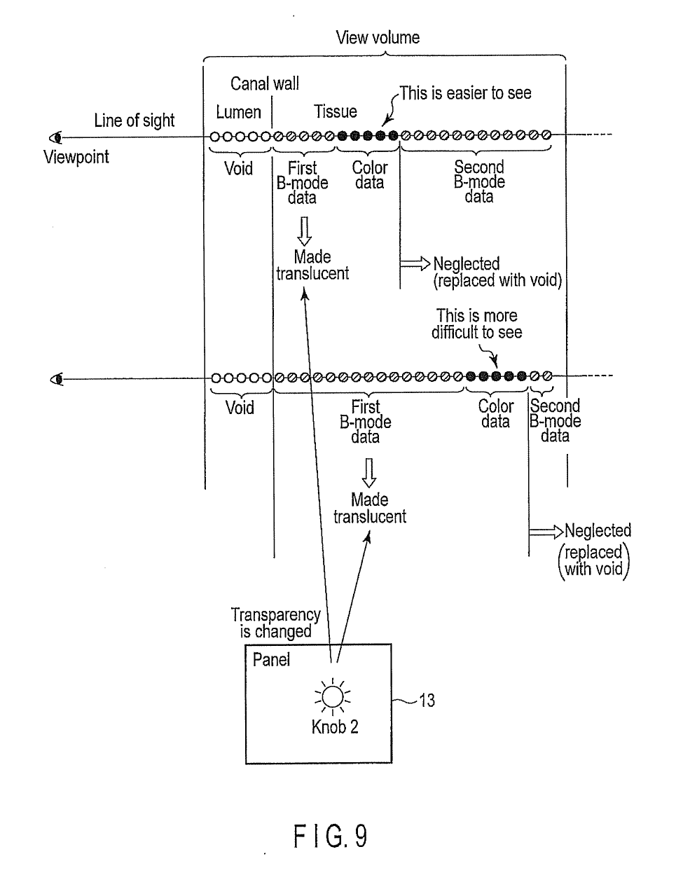

In rendering processing using opacities like those shown in FIG. 6, the larger the distance from the canal wall, the higher the influence of the B mode on the data, and the more difficult to see a blood flow image. In this case, in order to further improve the visibility, it is possible to automatically control the transparency (opacity) of the first B-mode data in accordance with a diagnosis region or manually control it by operating the knob of the input device 13, as shown in FIG. 9. That is, when the operator selects a predetermined region with a diagnosis selection switch (SW), the control processor 29 determines an opacity from the selected region and a prepared conversion table. Alternatively, when the operator changes the transparency by operating the knob, the control processor 29 determines an opacity corresponding to the transparency after the change, as shown in FIG. 9. The volume data generation unit 26 generates a virtual endoscopic image by executing rendering processing using the determined opacity.

### Second Modification

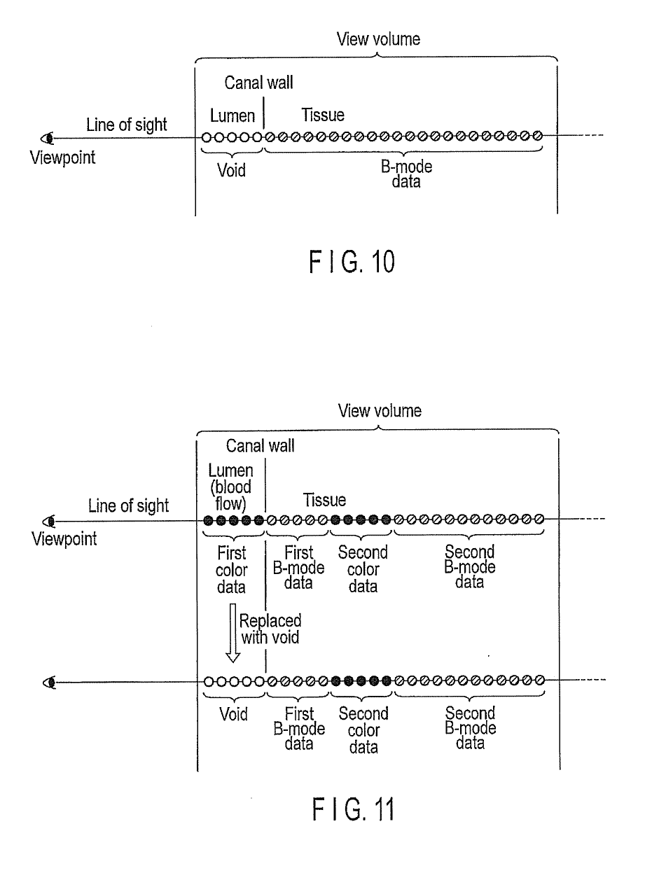

The above embodiment has exemplified the case in which each line of sight extends through a blood flow near the canal wall. As shown in FIG. 10, however, some line of sight may not extend through a blood flow in the tissue near the canal wall, with void data and B-mode data being arranged in the order named when viewed from the viewpoint. In this case, it is preferable to perform general volume rendering in the view volume by using B-mode data from the viewpoint. In this manner, the apparatus performs processing according to the above embodiment when a line of the respective lines of sight in a view volume extends through a blood flow in the tissue near the canal wall, and executes processing according to this modification when a line of sight of the respective lines of sight does not extend through the blood flow in the tissue near the canal wall. This makes it possible to properly generate and display a virtual endoscopic image including a blood flow near the canal wall buried in the tissue and greatly improve the diagnostic performance.

### Third Modification

The above embodiment has exemplified the case in which no blood flow exists in the lumen (void data exists on the nearest side to a viewpoint). In contrast to this, a blood flow sometimes exists in the lumen (color data sometimes exists on the nearest side to a viewpoint instead of void data). This modification will exemplify such a case.

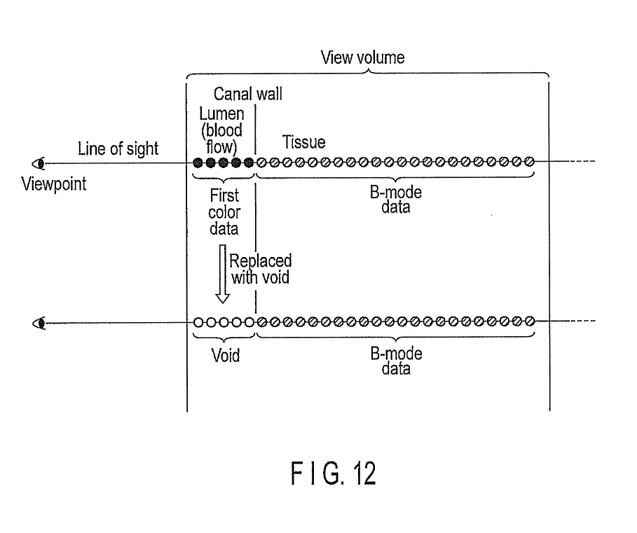

FIG. 11 shows a case in which a blood flow exists in the lumen (that is, the first color data exists in the lumen) and a line of sight extends through the second color data corresponding to the blood flow near the canal wall. FIG. 12 shows a case in which a blood flow exists in the lumen as in the above case, but a line of sight does not extend through the second color data corresponding to the blood flow near the canal wall. In the case shown in FIG. 11, in the view volume, the first color data, the first B-mode data, the second color data, and the second B-mode data are arranged in the order named when viewed from the viewpoint. In the case shown in FIG. 12, in the view volume, the first color data and the B-mode data are arranged in the order named when viewed from the viewpoint. In either of the cases, the arrangement order and position information of data are obtained. Therefore, the near-lumen blood flow extraction unit 27 can know the position information of the first color data when tracing from a viewpoint along a line of sight, by using the arrangement order and position information of data. Upon replacing the first color data with void data, the apparatus executes the same processing as that in step S4 described above. This can properly generate and display a virtual endoscopic image including a blood flow near the canal wall buried in the tissue regardless of the presence/absence of a blood flow in the lumen.

### Application Example

It is possible to set an MPR (Multi-Planar Reconstruction) slice and three orthogonal slices by using the virtual endoscopic image generated by the processing according to the above embodiment and automatically display images corresponding to the set slices. That is, the image processing unit 28 sets an MPR slice or three orthogonal slices in at least one of B-mode volume data and color volume data with reference to the viewpoint used in near-lumen blood flow extraction processing and an arbitrary point designated on a virtual endoscopic image. The image processing unit 28 generates an image corresponding to the MPR slice or the three orthogonal slices. The monitor 14 displays the generated tomogram together with, for example, a virtual endoscopic image in a predetermined form. Note that it is preferable to allow to rotate a set slice and arbitrarily control its position and direction relative to a virtual endoscopic image in accordance with instructions input from the input device 13.

### Effects

The above ultrasonic diagnostic apparatus determines the arrangement order of data viewed from a viewpoint on each line of sight in a view volume. When void data, B-mode data corresponding to the canal wall, and color data corresponding to a blood flow near the canal wall buried in the tissue are arranged in the order named from a viewpoint, the apparatus executes rendering upon replacing the B-mode data located nearer to the viewpoint than the color data with void data, and then generates and displays a virtual endoscopic image including the blood flow near the canal wall buried in the tissue. When the first color data corresponding to a blood flow in the lumen, B-mode data corresponding to the canal wall, and the second color data corresponding to a blood flow near the canal wall buried in the tissue are arranged in the order named from a viewpoint, the apparatus executes rendering upon, for example, replacing the first color data and the B-mode data with void data, and then generates and displays a virtual endoscopic image including the blood flow near the canal wall buried in the tissue. Therefore, the observer can visually recognize the blood flow near the canal wall existing in the canal wall easily and intuitively by observing a displayed virtual endoscopic image. This can greatly improve the diagnostic performance.

In addition, when color data corresponding to a blood flow near the canal wall buried in the tissue is located at a position sufficiently spaced apart from the canal wall, the apparatus generates and displays a virtual endoscopic image by using color data limited to an arbitrary distance from the canal wall. It is therefore possible to properly visualize blood flow information near the canal wall regardless of the size of the distribution region of color data corresponding to a blood flow near the canal wall buried in the tissue, thereby providing a high-quality diagnostic image.

Furthermore, when a line of sight does not extend through a blood flow near the canal wall buried in the tissue, this ultrasonic diagnostic apparatus performs general volume rendering by using B-mode data. This makes it possible to properly visualize the canal wall (canal tissue) itself when no blood flow information exists near the canal wall, and hence to provide a high-quality diagnostic image.

Note that the present invention is not limited to each embodiment described above, and constituent elements can be modified and embodied in the execution stage within the spirit and scope of the invention. The followings are concrete modifications.

(1) Each function associated with each embodiment can also be implemented by installing programs for executing the corresponding processing in a computer such as a workstation and expanding them in a memory. In this case, the programs which can cause the computer to execute the corresponding techniques can be distributed by being stored in recording media such as magnetic disks ((floppy®) disks, hard disks, and the like), optical disks (CD-ROMs, DVDs, and the like), and semiconductor memories.

(2) Each embodiment described above has exemplified the case in which processing is assumed to be performed inside the lumen, and perspective projection is used. However, without being limited to the above case, it is possible to use parallel projection with a viewpoint being set at infinity.

(3) The above embodiment has exemplified the case in which the ultrasonic data acquired by the ultrasonic diagnostic apparatus is used. However, without being limited to ultrasonic data, the technique according to the above embodiment can be applied to any three-dimensional image data including tissue data and blood flow data which are acquired by an X-ray computed tomography apparatus, magnetic resonance imaging apparatus, and X-ray diagnostic apparatus, and the like.

Various inventions can be formed by proper combinations of a plurality of constituent elements disclosed in the above embodiments. For example, several constituent elements may be omitted from all the constituent elements in each embodiment. In addition, constituent elements of the different embodiments may be combined as needed.

### REFERENCE SIGNS LIST

1 . . . ultrasonic diagnostic apparatus

12 . . . ultrasonic probe

13 . . . input device

14 . . . monitor

21 . . . ultrasonic transmission unit

22 . . . ultrasonic reception unit

23 . . . B-mode processing unit

24 . . . blood flow detection unit

25 . . . RAW data memory

26 . . . volume data generation unit

27 . . . near-lumen blood flow extraction unit

28 . . . image processing unit

29 . . . control processor

30 . . . display processing unit

31 . . . storage unit

32 . . . interface unit

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasonic diagnostic apparatus comprising:
a volume data acquisition unit configured to acquire first volume data corresponding to a three-dimensional region including a lumen of an object by scanning the three-dimensional region in a B mode with an ultrasonic wave and acquire second volume data by scanning the three-dimensional region in a blood flow detection mode with an ultrasonic wave;
a setting unit configured to set a viewpoint in the lumen, and a plurality of lines of sight with reference to the viewpoint;
a determination unit configured to determine a line of sight, on which tissue data corresponding to an outside of the lumen, and on which blood flow data corresponding to an outside of the lumen are arranged;
a control unit configured to control at least a parameter value corresponding to each voxel of the tissue data existing on the determined line of sight;
an image generation unit configured to generate a virtual endoscopic image based on the viewpoint by using the first volume data including voxels whose parameter values are controlled and the second volume data; and
a display unit configured to display the virtual endoscopic image.

2. The ultrasonic diagnostic apparatus according to claim 1, wherein when blood flow data exists as data corresponding to the intraluminal region, the control unit controls at least a parameter value attached to each voxel of data corresponding to the intraluminal region.

3. The ultrasonic diagnostic apparatus according to claim 2, wherein the control unit controls a parameter value attached to each voxel of a region corresponding to data corresponding to the intraluminal region so as to make a blood flow in the lumen become transparent or translucent.

4. The ultrasonic diagnostic apparatus according to claim 1, wherein the control unit controls a parameter value attached to each voxel of a region corresponding to the tissue data so as to make a region corresponding to the tissue data become transparent or translucent.

5. The ultrasonic diagnostic apparatus according to claim 4, wherein the control unit controls a parameter value attached to each voxel of a region corresponding to the tissue data by using a transparency or an opacity set in accordance with a diagnosis region or an input from an input unit.

6. The ultrasonic diagnostic apparatus according to claim 3, wherein the control unit controls a parameter value attached to each voxel of a region corresponding to data corresponding to the intraluminal region by using a transparency or an opacity set in accordance with a diagnosis region or an input from an input unit.

7. The ultrasonic diagnostic apparatus according to claim 1, wherein the image generation unit generates the virtual endoscopic image upon excluding data located at a position deeper than the blood flow data when viewed from the viewpoint.

8. The ultrasonic diagnostic apparatus according to claim 1, wherein the image generation unit generates the virtual endoscopic image upon excluding the blood flow data located at a position deeper than a predetermined distance from a boundary between the inside of the lumen and the tissue data.

9. The ultrasonic diagnostic apparatus according to claim 1, wherein the image generation unit generates the virtual endoscopic image by rendering processing using perspective projection.

10. The ultrasonic diagnostic apparatus according to claim 1, wherein the image generation unit generates the virtual endoscopic image by volume rendering.

11. The ultrasonic diagnostic apparatus according to claim 1, wherein the image generation unit sets at least one slice for at least one of the first volume data and the second volume data with reference to the viewpoint and an arbitrary point designated on the virtual endoscopic image, and generates a tomogram corresponding to at least the one slice, and
the display unit displays the tomogram and the virtual endoscopic image.

12. An ultrasonic image processing apparatus comprising:
a volume data storage unit configured to store first volume data acquired by scanning a three-dimensional region including a lumen of an object in a B mode with an ultrasonic wave and second volume data acquired by scanning the three-dimensional region in a blood flow detection mode with an ultrasonic wave;
a setting unit configured to set a viewpoint and a plurality of lines of sight with reference to the viewpoint in the lumen;
a determination unit configured to determine a line of sight, of the plurality of lines of sight, on which tissue data corresponding to an outside of the lumen and blood flow data corresponding to a blood flow outside the lumen are arranged;
a control unit configured to control at least a parameter value attached to each voxel of the tissue data existing on the determined line of sight;
an image generation unit configured to generate a virtual endoscopic image based on the viewpoint by using the first volume data including voxels whose parameter values are controlled and the second volume data; and
a display unit configured to display the virtual endoscopic image.

13. The ultrasonic image processing apparatus according to claim 12, wherein when blood flow data exists as data corresponding to the intraluminal region, the control unit controls at least a parameter value attached to each voxel of data corresponding to the intraluminal region.

14. The ultrasonic image processing apparatus according to claim 13, wherein the control unit controls a parameter value attached to each voxel of a region corresponding to data corresponding to the intraluminal region so as to make a blood flow in the lumen become transparent or translucent.

15. The ultrasonic image processing apparatus according to claim 12, wherein the control unit controls a parameter value attached to each voxel of a region corresponding to the tissue data so as to make a region corresponding to the tissue data become transparent or translucent.

16. The ultrasonic image processing apparatus according to claim 15, wherein the control unit controls a parameter value attached to each voxel of a region corresponding to the tissue data by using a transparency or an opacity set in accordance with a diagnosis region or an input from an input unit.

17. The ultrasonic image processing apparatus according to claim 14, wherein the control unit controls a parameter value attached to each voxel of a region corresponding to data corresponding to the intraluminal region by using a transparency or an opacity set in accordance with a diagnosis region or an input from an input unit.

18. The ultrasonic image processing apparatus according to claim 12, wherein the image generation unit generates the virtual endoscopic image upon excluding data located at a position deeper than the blood flow data when viewed from the viewpoint.

19. The ultrasonic image processing apparatus according to claim 12, wherein the image generation unit generates the virtual endoscopic image upon excluding the blood flow data located at a position deeper than a predetermined distance from a boundary between the inside of the lumen and the tissue data.

20. The ultrasonic image processing apparatus according to claim 12, wherein the image generation unit generates the virtual endoscopic image by rendering processing using perspective projection.

21. The ultrasonic image processing apparatus according to claim 12, wherein the image generation unit generates the virtual endoscopic image by volume rendering.

22. The ultrasonic image processing apparatus according to claim 12, wherein the image generation unit sets at least one slice for at least one of the first volume data and the second volume data with reference to the viewpoint and an arbitrary point designated on the virtual endoscopic image, and generates a tomogram corresponding to at least the one slice, and
the display unit displays the tomogram and the virtual endoscopic image.

23. An ultrasonic image processing method which uses first volume data acquired by scanning a three-dimensional region including a lumen of an object in a B mode with an ultrasonic wave and second volume data acquired by scanning the three-dimensional region in a blood flow detection mode with an ultrasonic wave, comprising:
setting a viewpoint and a plurality of lines of sight with reference to the viewpoint in the lumen;
determining a line of sight, of the plurality of lines of sight, on which tissue data corresponding to an outside of the lumen and blood flow data corresponding to a blood flow outside the lumen are arranged;
controlling at least a parameter value attached to each voxel of the tissue data existing on the determined line of sight;
generating a virtual endoscopic image based on the viewpoint by using the first volume data including voxels whose parameter values are controlled and the second volume data; and
displaying the virtual endoscopic image.
